(19) United States
(12) Patent Application Publication
(10) Pub. No.: US 2026/0020863 A1
(43) Pub. Date: Jan. 22, 2026

(54) COMPACT TOURNIQUET

(71) Applicant: Frank Milton Barnett, New Waverly, TX (US)

(72) Inventor: Frank Milton Barnett, New Waverly, TX (US)

(21) Appl. No.: 19/270,380

(22) Filed: Jul. 15, 2025

Related U.S. Application Data
(60) Provisional application No. 63/672,244, filed on Jul. 16, 2024.

(51) Int. Cl. A61B 17/132; A44B 11/00; A45C 11/00; A61B 90/96

(52) U.S. Cl. CPC A61B 17/1325; A44B 11/006; A45C 11/002; A61B 90/96

(57) ABSTRACT

Various embodiments of a compact tourniquet, both with and without integrated buckle, loop, and locking mechanism, are disclosed. In one or more embodiments, an elongate compression strap formed of a strip of fabric characterized by a low-coefficient of friction and high tensile strength for providing occlusion of blood while remaining small and discreet. A turnbuckle or other tensioning device may be provided, which may be integrated into another device or item. The compact tourniquet may be stored or integrated into a cell phone case, a key chain, a hat band, waist band, belt, wallet, pistol handgrip, holster, or rifle stock. It is possible to integrate one or more compact tourniquet embodiments disclosed herein effectively into most any item—boots, ties, pants hems, glasses keepers, lifejackets, et cetera—because the design of the tourniquet cleat can be different and unique for each item so as to blend into the design of the item into which the tourniquet is integrated.

### CROSS REFERENCE TO RELATED APPLICATION

This non-provisional application claims priority under 35 USC 119(e) to provisional application U.S. 63/672,244 filed Jul. 16, 2024 and entitled “Compact Tourniquet”, which is incorporated herein by reference.

### BACKGROUND

According to recent studies of trauma events, when a person experiences serious blood loss from an injury to an extremity, death often results within four minutes. The injured person's odds of surviving increase 600% if a tourniquet is correctly applied by the injured person or an immediate responder, that is, a person who is with the injured party when the injury is sustained or is present there within the immediate aftermath of the injury. Unfortunately, first responders (police, fire, and EMS) average over seven minutes response times.

Recognizing the need for faster bleeding control, the national “Stop the Bleed” training program trains citizens to apply tourniquets and recommends that individuals, organizations and businesses keep tourniquets near at hand. Many locations now have “Stop the Bleed” lockers containing tourniquets at publicly accessible locations. However, the probability of such a locker being nearby to an injury site is still remote. Moreover, for individuals that are in a public place that has a “Stop the Bleed” locker containing tourniquets, it is unlikely that they would know that tourniquets are available, where they are located, or how to use or apply them.

While in some rare instances individuals may carry a tourniquet in their vehicle, they, with the exception of on-duty policemen, firefighters and EMS personnel, almost never carry one on their person, because available tourniquets are bulky and a burden to carry at all times.

Accordingly, it would be advantageous to equip most people with a tourniquet that can be readily and easily carried at all times without forethought and the instructions or training to immediately use it should the unfortunate need suddenly and unexpectedly arise.

Several types of tourniquets are now widely available: Examples of the largest, a strap and windlass type with a 1½″ or wider strap, are the CAT (U.S. Pat. Nos. 7,842,067 and 7,892,253) and the SoffT, and equivalents thereof. These are the type used by military and law enforcement, and they are considered the standard. However, they are fairly bulky. The CAT uses a compression strap that slides within a wider and thicker, belt-like sleeve; this arrangement makes the unsuitable for smaller children, because it cannot tighten adequately around a small limb. A more compact choice is an approximate 4″ wide rubber band type, such as the Swat-T (U.S. Pat. No. D733306). The most compact is the R.A.T. (U.S. Pat. No. 9,168,044), the strap of which is a very narrow stretchable cord. While the R.A.T. is compact, it lacks the strap width and mechanical tensioning device, and it is considered to be less effective than the tourniquets previously mentioned.

Although other makeshift tourniquets may be fashioned during an emergency, which may be considered compact, such are unlikely to approach or meet the standards of Tactical Combat Casualty Care. It is desirable, therefore, to provide an “always-carried” tourniquet that approaches or meets standards of Tactical Combat Casualty Care.

### BRIEF DESCRIPTION OF THE DRAWINGS

Many aspects of the present disclosure can be better understood with reference to the following drawings, in which:

FIG. 1 is a perspective view of a compact tourniquet assembly according to one or more embodiments;

FIG. 2 is an elevation view of the front side of the compact tourniquet assembly of FIG. 1;

FIG. 3 is an elevation view of the front side of the compact tourniquet assembly of FIG. 1;

FIG. 4 is a perspective view of the front side of an integrated compact tourniquet/cellular phone case assembly according to one or more embodiments, showing a folded compression strap located within the case;

FIG. 5 is a perspective view of the back side of the integrated compact tourniquet/cellular phone case assembly of FIG. 4, showing a turnbuckle, strap backing pad and buckle forming a removable portion of the cellular phone case;

FIG. 6 is a perspective view of the back side of the integrated compact tourniquet/cellular phone case assembly according to one or more embodiments, showing a removable kickstand that deploys a tourniquet from the phone case while the phone remains housed therein;

FIG. 7 is a perspective view of the deployed tourniquet of the integrated compact tourniquet/cellular phone case assembly of FIG. 6;

FIGS. 8 and 9 are plan views of the back sides of the integrated compact tourniquet/cellular phone case assemblies according to various embodiments, showing alternative locations for various tourniquet components;

FIG. 10 is a perspective view of a compact tourniquet assembly according to one or more embodiments, in which the compression strap is equipped with slits or holes for initial securing of the strap to the turnbuckle prior to tensioning with the turnbuckle; and

FIG. 11 illustrate perforated indicia for marking the time of tourniquet application that may be disposed on tourniquets, showing hour and quarter hour marks.

The components in the drawings are not necessarily to scale, with emphasis instead being placed upon clearly illustrating the principles of the disclosure. Moreover, in the drawings, like reference numerals designate corresponding parts throughout the several views.

### DETAILED DESCRIPTION

Various embodiments of a compact tourniquet, both with and without integrated buckle, loop, and locking mechanism, are disclosed. In one or more embodiments, the compact tourniquet may be stored or integrated into a cell phone case, with the concomitant advantage that most people carry their cell phones with them at all times—there is no need to remember to add another item to one's pocket or purse. Moreover, having a tourniquet integrally located with a cell phone provides economy of action: The phone will be used first to call 911, then the tourniquet is deployed from case. As described in greater detail hereinafter, a QR code may be provided on the tourniquet or its packaging, which may also be scanned using the cell phone to provide instructions for proper use of the tourniquet, should it be necessary.

In other embodiments, the compact tourniquet may be stored or integrated into a key chain, a hat band, waist band, belt, wallet, pistol handgrip, holster, or rifle stock. Indeed, it is possible to integrate one or more compact tourniquet embodiments disclosed herein effectively into most any item—boots, ties, pants hems, glasses keepers, lifejackets, et cetera—because the design of the tourniquet cleat can be different and unique for each item so as to blend into the design of the item into which the tourniquet is integrated. Accordingly, tourniquets may be made readily available anywhere they may be needed.

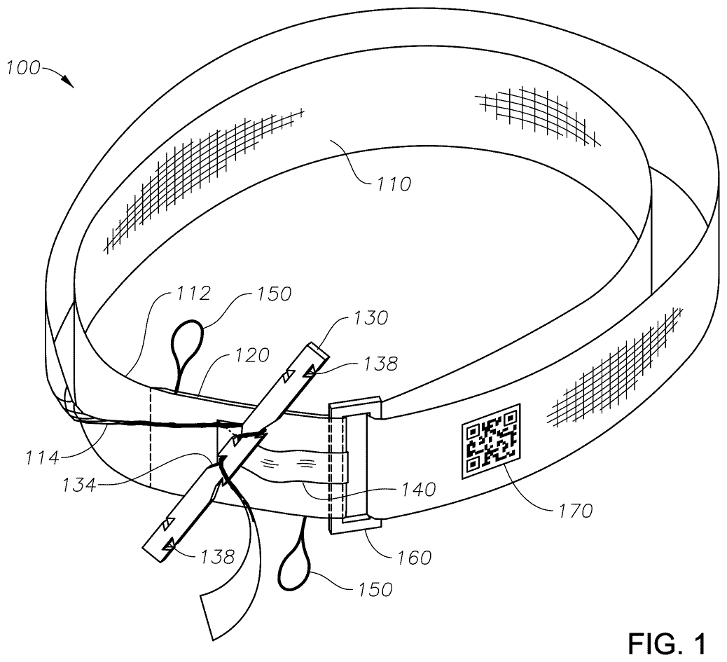

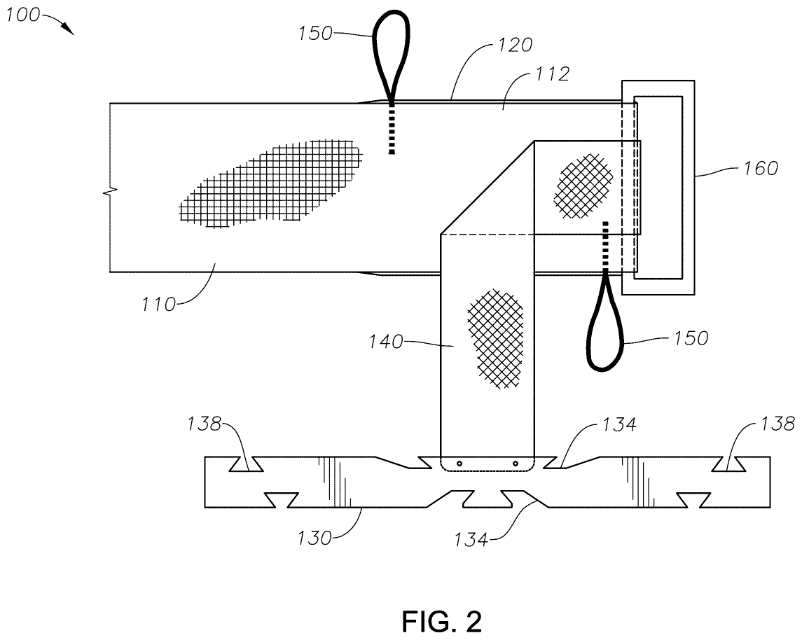

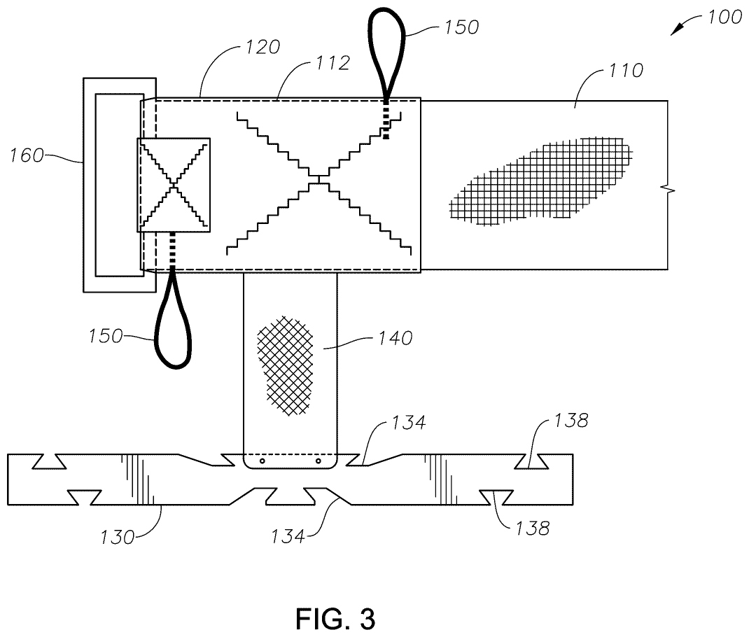

FIG. 1 is a perspective view, FIG. 2 is an elevation view of the front, or outer side, and FIG. 3 is an elevation view of the back, or inner side, respectively, of a compact tourniquet assembly 100 according to a first embodiment. Referring to FIGS. 1-3, tourniquet assembly 100 includes a compression strap 110 which is preferably constructed of one or more fabric strips, approximately between 32 and 38 inches long and 1¼ to 2 inches wide. A first end 112 of compression strap 110 is attached to a short strip of strap backing 120 by sewing, welding, glueing or a combination of these or other mechanical attachment.

Tourniquet assembly 100 further includes a turnbuckle 130 with cleating profile 134, a turnbuckle attachment strap 140, a turnbuckle keeper 150, and a buckle 160. In one or more embodiments, turnbuckle 130, turnbuckle attachment strap 140, turnbuckle keeper 150, and buckle 160 are attached or readily attachable to strap backing 120.

Compression strap 110 is preferably constructed from Dyneema® composite fabric with polyethylene backing or other material with similar characteristics of strength, low stretch, low friction coefficient, and extreme thinness. The extremely thin nature of this material is a characteristic that departs substantially from prior art and is the physical characteristic that enables construction of a functional strap-type emergency tourniquet that allows integration into everyday carry items such as a cell phone case, small tube, wallet, hatband, et cetera. The thinness, in conjunction with the strength of the Dyneema® composite fabric material, also allows the strap to be twisted substantially, forming a string-like portion 114 of compression strap 110 at its second, bitter end that can be easily attached to turnbuckle cleat 134. The low friction coefficient of compression strap 110 also departs substantially from prior art in that, when circumferentially surrounding a body part and tensioned against itself, it slides against another portion of the strap 110 and requires less tensioning force (such as from manipulation of turnbuckle 130) to achieve sufficient compression force for the occlusion of blood.

There may be other suitable materials, but the preferred strap material is Dyneema® composite fabric. For the thinner weight fabrics, such as 0.51 oz or 0.67 ounce variants, some cross-strap bracing (by stitching or other material) may be desirable to keep compression strap 110 from bunching up and narrowing during compression. The 0.8 or 1.0 ounce thickness is better at controlling cross-strap deformation, thereby maintaining the width of compression strap 110 while it is under tension, particularly when applying tourniquet 100 over clothing.

Strap backing 120 defines a flexible base for compression strap 110. Strap backing 120 is ideally constructed from flexible rubber, plastic, silicone or like material to which the first end of compression strap 110 is attached. Strap backing 120 provides thicker material under the location of tensioning turnbuckle 130 and has a greater friction coefficient than that of compression strap 110, resulting in less likelihood of the entire tourniquet assembly 100 turning against the applied body part.

In a preferred embodiment, turnbuckle 130 is an elongate member constructed from hard plastic, graphite, metal or a composite of sufficient length (between about 3½ to 6 inches) to provide necessary mechanical leverage for tightening compression strap 110 during application of tourniquet 100. Turnbuckle 130 is attached at its center to turnbuckle attachment strap 140—a short length of strap ideally of the same or similar material as the compression strap 110. Turnbuckle 130 is equipped with grooves and/or cleat features 134 that allow the loose end 114 of compression strap 110 to be gathered and twisted onto cleat profile 134 to secure compression strap 110 to turnbuckle 130 for tensioning thereof by rotation of the turnbuckle. Turnbuckle 130 includes notches 138 or similar profile or features at both distal ends for the attachment of a turnbuckle keeper 150.

According to one or more embodiments, turnbuckle attachment strap 140 is constructed from Dyneema® or material with similar characteristics. Turnbuckle attachment strap 140 is attached at its first end to strap backing 120 near buckle 160 and at its second end to a center portion of turnbuckle 130 by sewing, welding, gluing, a combination of these, or other mechanical or chemical attachment methods, as understood by routineers in the art. As turnbuckle 130 is rotated during application of tourniquet assembly 100, turnbuckle attachment strap 140 will readily twist and wind up into a string-like profile.

Turnbuckle keeper 150 provides a means to secure turnbuckle 130 in place after tourniquet tensioning. In one or more embodiments, turnbuckle keeper 150 consists of a pair of loops of thin cord or string attached by mechanical or chemical attachment methods between compression strap 110 and strap backing 120 near diagonally opposite corners of strap backing 120. Turnbuckle keeper 150 loops are of sufficient length to just reach the attachment points 138 on turnbuckle 130 when the turnbuckle is in a position near parallel to compression strap 110.

Buckle 160 is preferably constructed from hard plastic, polymer, graphite, metal or a composite material. In some embodiments, buckle 160 is permanently attached to compression strap 110 and strap backing 120 at the first end 112 of compression strap 110. In other embodiments, buckle 160 has a rectangular “S” shape, and one end of buckle 160 may be removably inserted through a sleeve formed in a hem of compression strap 110 or between compression strap 110 and strap backing 120. In other embodiments, buckle 160 is formed of a thin rigid rod held in place within a hem formed perpendicular to and at the end of the strap backing; a transverse slot formed through the strap backing adjacent the hem completes the buckle and allows the second end of the compression to be passed through and taken up. In still other embodiments, buckle 160 is constructed entirely of fabric. For example, buckle 160 as shown in FIG. 7 is constructed entirely of relatively stiff strap backing 120 with a transverse slot formed therethrough.

In operation, tourniquet assembly 100 is deployed as follows: Strap backing 120 is placed against the wounded appendage of the injured person. The second, bitter end 114 of compression strap 110 is wrapped about the limb, fed through buckle 160, and wrapped back about the limb in the opposite direction. The bitter end 114 of compression strap 110 is then twisted as necessary to pass through the cleating profile 134 of turnbuckle 130. Turnbuckle 130 is then rotated to compress compression strap 110 until adequate occlusion of blood from the wound is obtained. Then, with turnbuckle 130 positioned nearly parallel to compression strap 110, turnbuckle keeper loops 150 are stretched and hooked into notches 138 of turnbuckle 130, thereby holding turnbuckle 130 and preventing it from unwinding.

In one or more embodiments, tourniquet 100 includes a Quick Response (“QR”) code 170. The QR code may be scanned to open a website that may provide instructions on use of the tourniquet. Additionally, the web site may allow one to report important information that can be accessed by medical personnel, such as date and time the tourniquet was applied, conditions of the patient, name of person applying the tourniquet, the type of tourniquet, effectiveness of tourniquet, type of injury, et cetra. Such use of QR code 170 and its associated website, could allow for further refinement of tourniquet and be used to laude the names of heros saving lives and promote tourniquet use education.

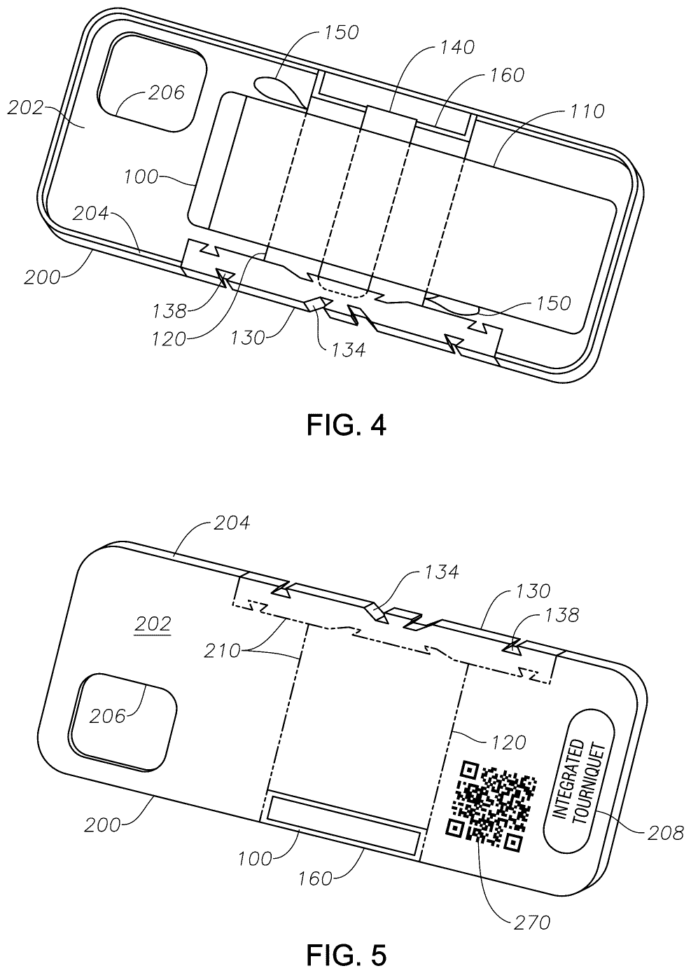

In one or more embodiments, compact tourniquet assembly 100 of FIGS. 1-3 is integrated into a cellular telephone case. In such embodiments, FIGS. 1-3 illustrate tourniquet assembly 100 detached from the phone case, and FIGS. 4 and 5 illustrate tourniquet assembly 100 integrated into a phone case 200. FIG. 4 is a perspective view of the interior of phone case 200, and FIG. 5 is a perspective view of the exterior back side of phone case 200.

Referring to FIGS. 4 and 5, cell phone case 200 may be made of flexible rubber, plastic, silicone or like material, or a combination thereof. As is typical of many cell phone cases, case 200 includes a planar rectangular back surface 202 with radiused corners. Back surface 202 is bounded by lipped walls 204 which wrap around the sides of a cell phone (not illustrated) to removably secure the cell phone within the case. Back surface 202 may include an aperture 206 to accommodate cell phone camera lenses. Walls 204 may include other openings (not illustrated) for charging cables, speakers, microphones, and the like.

In one or more embodiments, shown most clearly in FIG. 5, strap backing 120, turnbuckle 130, and buckle 160 of tourniquet assembly 100 form an integral portion of back surface 202 and walls 204 of case 200. Using strap backing 120 formed of a flexible silicone back of phone case 200 against the limb also works to help maintain the width of compression strap 100 under tension. Preferably, an indicium, placard, or other marking 208 designating the integration of a tourniquet into the item would be clearly located on cell phone case 200 (or any other item into which a compact tourniquet according to one or more embodiments may be integrated). Similarly, cell phone case 200 may include a QR code 270, as described above with respect to QR code 170 of FIG. 1.

Strap backing 120, turnbuckle 130, and buckle 160 may be removed from the remainder of case 200 for deployment. As illustrated, they may be broken apart from the remaining portions along perforations 210 formed in phone case 200. Alternatively, strap backing 120, turnbuckle 130, and buckle 160 may be independent discrete components of phone case 200 and may be snapped in their respective positions and held be detents, for example.

As shown in FIG. 4, compression strap 110, turnbuckle attachment strap 140, and turnbuckle keeper 150 are disposed within the interior of phone case 200 and fit comfortably behind a cell phone. For compression strap 110 to fit in a typical-sized phone case while adding minimal or no thickness to the phone case, strap 110 must be very thin. A 1.5 inch wide strip of Dyneema® composite fabric, about 37″ long, can be folded to fit behind the phone in phone case 200 causing no noticeable difference in the fit of the phone as verified using typical off-the-shelf phone cases.

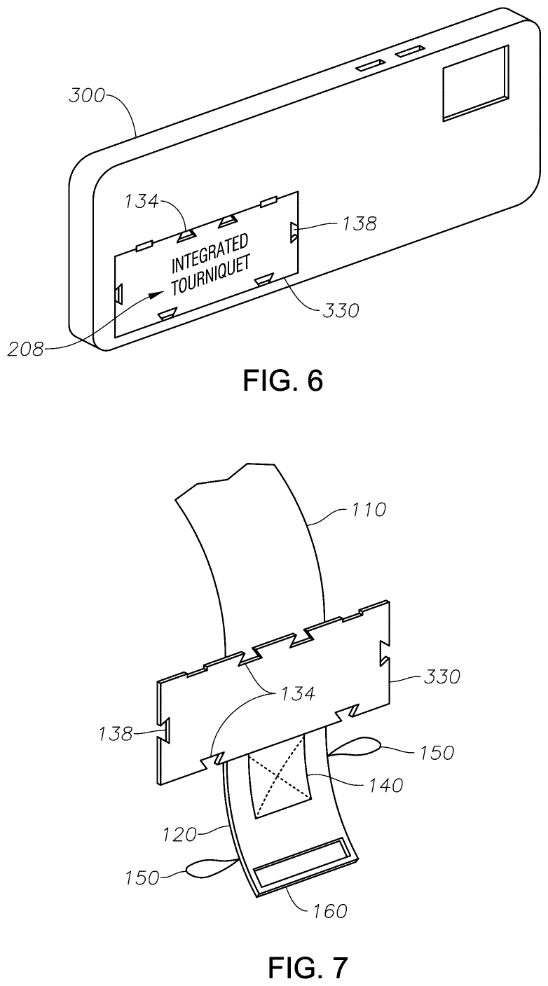

There are alternative embodiments of integration of a compact tourniquet into a cell phone case. For example, FIGS. 6 and 7 detail an embodiment in which a portion of a rigid cell phone case 300, such as a hinged kickstand 330 (for holding the phone near in landscape orientation nearly vertically for viewing videos, for example), may separated from case 300 and pulled, deploying the tourniquet 100′ from case 300 while the case still contains the phone. Tourniquet 100′ is substantially the same as tourniquet 100 of FIGS. 1-3, having compression strap 110, strap backing 120, turnbuckle attachment strap 140, turnbuckle keeper 150 and buckle 160. Kickstand 330 forms the turnbuckle of tourniquet 100′. As with turnbuckle 130 of FIGS. 1-3, kickstand 330 includes cleating profile 134 and keeper profile 138.

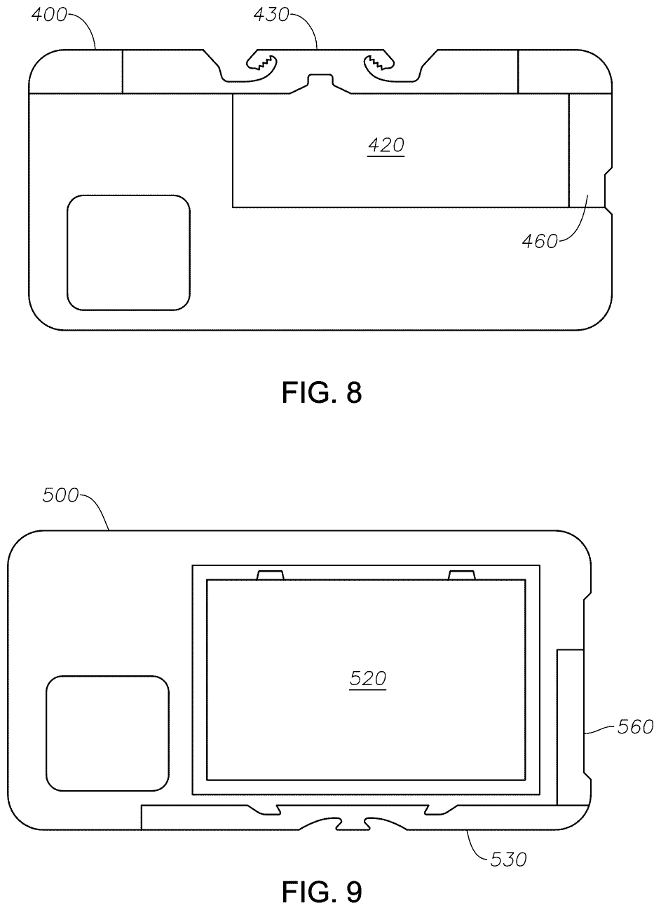

FIGS. 8 and 9 illustrate alternative integrated compact tourniquet/cellular phone case assembly embodiments 400, 500 and show various places on the case where strap backing 420, 520, turnbuckle 430, 530, and buckle 460, 560, respectively, may be located. Other suitable locations may occur to those skilled in the art and are covered within the scope of this disclosure.

Although not illustrated, in one or more embodiments, all of the portions of the compact tourniquet as disclosed herein are stored within a cell phone case. The phone case remains useable after the tourniquet is removed, allowing practice with the tourniquet and replacement in the phone case and without affecting the future use of the case or tourniquet.

Dyneema® composite fabric also has a thin polyester film, creating a low friction surface that when used as the compression strap enables the second wrap of tourniquet to turn on the first (against the limb) wrap. In one or more embodiments, such as where the entire tourniquet is disclosed inside of a cell phone case behind the phone, a tourniquet assembly employing Dyneema® composite fabric for the compression strap may allow a user to apply the desired pressure (>160 mm/Hg) without an exterior mechanical tensioning device such as a turnbuckle, windlass, knob, or cam. This embodiment, without the exterior mechanical tensioning device, allows the compact tourniquet to be integrated with countless items. For example, a tourniquet may be integrated in a hat band, a belt, the waistband of boardshorts, a wallet, a purse, inside of a pistol grip, a holster, and in a number of areas on a AR platform rifle. The tourniquet may be folded between a patch or emblem, such as might be worn on a uniform, and the patch's Velcro backing. In yet another embodiment, a compact tourniquet can be rolled up tightly and inserted into a tube, with a hard plastic buckle or windlass, if necessary, forming the body of a tube. The tube can be integrated with a flashlight, keychain, lighter, pepper spray cannister, laser pointer, pill holder, or other similar items or a combination thereof. The compact tourniquet as disclosed herein may provide for the widespread availability of tourniquets and, concomitantly, persons familiar with their use.

Although tourniquet 100 disclosed herein employs a turnbuckle tension device, the turnbuckle method of tensioning is only one method that can be used with a tourniquet or tourniquet/phone case combination. Other embodiments could employ different methods of tensioning such as knob, ratchet, screw, worm gear, et cetera, as known in the art. Such other mechanical tension devices are within the scope of the present disclosure.

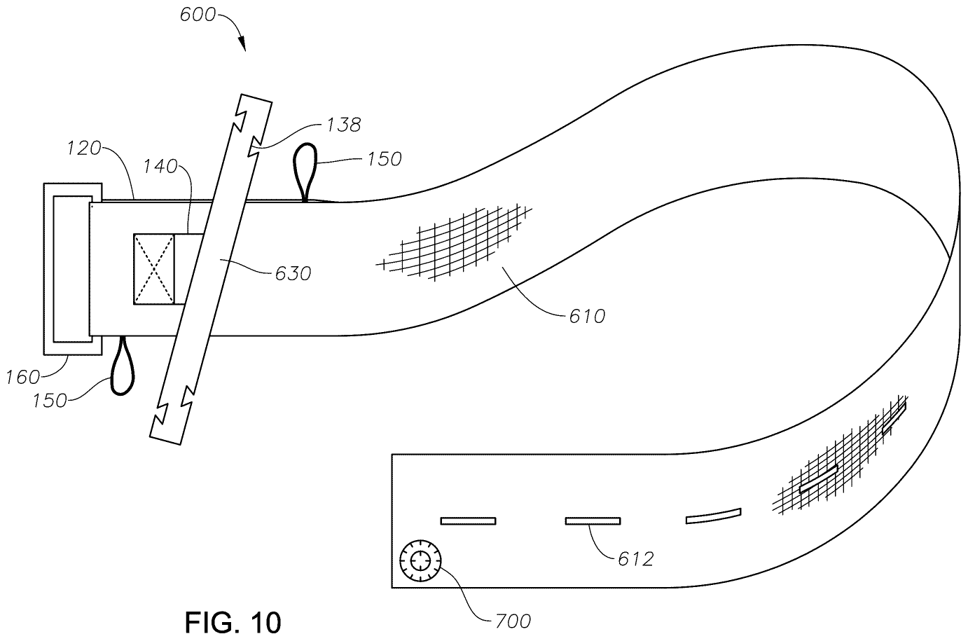

FIG. 10 is a perspective view of a compact tourniquet assembly 600 according to one or more embodiments, in which the compression strap 610 is equipped with slits or holes 612 for initial securing of compression strap 610 to the turnbuckle prior to tensioning with the turnbuckle. Tourniquet assembly 600 is substantially similar to tourniquet assembly 100 of FIGS. 1-3, having a compression strap 610 made of Dyneema® composite fabric like compression strap 110, a strap backing 120, turnbuckle attachment strap 140, turnbuckle keeper 150, and buckle 160. Likewise, turnbuckle 630 is similar to turnbuckle 130, having keeper notches 138 but omitting cleating profile 134. Instead of hooking the compression strap around a cleating profile, as in tourniquet 100, one end of turnbuckle 630 is passed through the closest hole or slit 612 of compression strap 610. Thereafter, the turnbuckle is tightened to occlude blood flow and then secured by hooking turnbuckle keeper loops 150 around keeper notches 138.

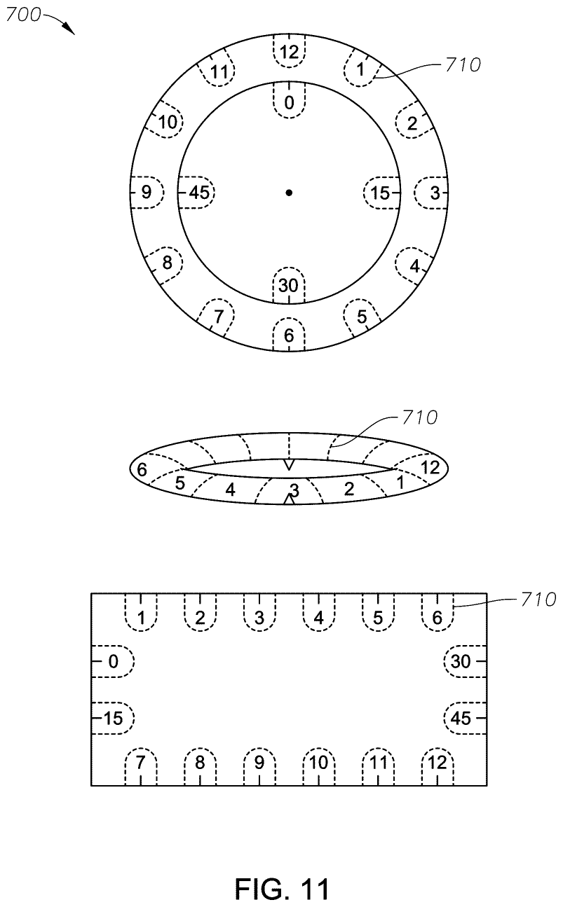

It is important for medical personnel to know the time of application of a tourniquet. Accordingly, in one or more embodiments an application time indicium may be provided on the tourniquet. Although indicium 700 is shown only on tourniquet 600 of FIG. 10, it is to be understood that an application time indicium may be provided on all tourniquet embodiments. FIG. 11 discloses three application time indicia 700 according to various embodiments which may be included on a tourniquet as a tag, decal, patch or the like. Indicia 700 include perforated portions which my be torn, punched, dimpled or otherwise marked, that indicated the hour and quarter hour when the tourniquet is applied. The time of application may be indicated on the tourniquet by writing the time on the body of the tourniquet, but if no writing method is available, by tearing or otherwise indicating at perforated portions of indicia 700. Preferably, indicia 700 are made from wax paper or other similar material that can be torn with little effort, but with sufficient strength and fluid resistance to perform their function.

It will be understood from the foregoing description that modifications and changes may be made in various embodiments of the present invention without departing from its true spirit. The descriptions in this specification are for purposes of illustration only and are not to be construed in a limiting sense.

The Abstract of the disclosure is solely for providing the a way by which to determine quickly from a cursory reading the nature and gist of technical disclosure, and it represents solely one or more embodiments.

What is claimed:

1. A compact tourniquet, comprising:
an elongate compression strap formed of a strip of thin fabric characterized by a low-coefficient of friction and high tensile strength and the ability to be twisted into thin string-like shape, said compression strap defining first and second distal ends;
a strap backing attached to said first end of said compression strap;
a turnbuckle defining a profile designed and arranged for capturing said second end of said compression strap;
a turnbuckle attachment strap coupled between said turnbuckle and said strap backing; and
a buckle coupled to said strap backing, whereby
said second end of said compression strap is operable to be passed through said buckle, folded back and captured by said profile of said turnbuckle;
said turnbuckle is operable to be wound with said second end of said compression strap captured by said profile to tension said tourniquet; and
said turnbuckle keeper is operable to secure said turnbuckle in said wound state.

2. The tourniquet of claim 1, wherein:
said compression strap is formed of Dyneema composite fabric.

3. The tourniquet of claim 1, wherein:
said compression strap has a length ranging from 32 to 38 inches long and a width ranging between 1¼ to 2 inches.

4. The tourniquet of claim 1, wherein:
said strap backing is formed of a flexible material characterized by a coefficient of friction substantially greater than the coefficient of friction of said compression strap.

5. The tourniquet of claim 1, further comprising:
a notch formed within said turnbuckle and dimensioned to capture said turnbuckle keeper.

6. The tourniquet of claim 1, further comprising:
a plurality of slots longitudinally formed in said compression strap, each slot dimensioned for receiving said profile of said turnbuckle therein to capture second end of said compression strap by said profile for tensioning said tourniquet.

7. The tourniquet of claim 1, further comprising:
a cleat formed in said profile of said turnbuckle dimensioned to capture second end of said compression strap by said profile for tensioning said tourniquet.

8. The tourniquet of claim 1, further comprising:
a cellular telephone case, said turnbuckle forming a removable portion of said case, said compression strap being foldable within an interior space defined by said case.

9. The tourniquet of claim 1, further comprising:
indicia disposed on said case for providing instruction on tourniquet use.

10. The tourniquet of claim 1, further comprising:
an indicium disposed on said compression strap for noting time of tourniquet application.

11. The tourniquet of claim 1, further comprising:
a turnbuckle keeper coupled to said strap backing.

12. A compact tourniquet, comprising:
an elongate compression strap formed of a strip of thin fabric characterized by a low-coefficient of friction and high tensile strength and the ability to be twisted into thin string-like shape, said compression strap defining first and second distal ends;
a strap backing attached to said first end of said compression strap;
a buckle coupled to said strap backing, whereby
said second end of said compression strap is operable to be passed through said buckle, folded back and tensioned with a second wrap of said compression strap about a limbs.

13. The tourniquet of claim 12, further comprising:
a tensioning device designed and arranged for capturing and tensioning said second end of said compression strap; and
a locking mechanism designed and arranged for maintaining said tensioning device in a tensioned state.

14. The tourniquet of claim 12, wherein:
said tourniquet is integrated with one of the group consisting of a hat band, a belt, a waistband, a wallet, a purse, a pistol grip, a holster, an article of clothing, a life vest, a patch, and a rifle.

15. The tourniquet of claim 12, further comprising:
a tube, said compression strap rolled and inserted into said tube.

16. The tourniquet of claim 15, wherein:
said tube is integrated with one of the group consisting of a flashlight, a keychain, a lighter, a spray cannister, a laser pointer, a pill holder, a safety device, and a rifle accessory.

17. The tourniquet of claim 15, further comprising:
indicia disposed on said tube for providing instruction on tourniquet use.

18. The tourniquet of claim 12, wherein:
said compression strap is formed of Dyneema composite fabric.

19. A cellular telephone case, comprising:
a case defining a case interior dimensioned for receiving a cellular telephone therein, a portion of said case being removable from the remainder of the case, said portion defining a turnbuckle;
an elongate compression strap formed of a strip of fabric characterized by a low-coefficient of friction and high tensile strength, said compression strap defining first and second distal ends, said compression strap being foldable into said interior space behind said cellular telephone;
a strap backing attached to said first end of said compression strap;
said turnbuckle defining a profile designed and arranged for capturing said second end of said compression strap;
a turnbuckle keeper coupled to said strap backing; and
a buckle coupled to said strap backing, whereby
said compression strap, said turnbuckle, said turnbuckle keeper and said bucket are operable to be removed from said remainder of said case;
said compression strap is operable to be disposed about a limb;
said second end of said compression strap is operable to be passed through said buckle, folded back and captured by said profile of said turnbuckle;
said turnbuckle is operable to be wound with said second end of said compression strap captured by said profile to tension said tourniquet; and
said turnbuckle keeper is operable to secure said turnbuckle in said wound state.

20. The tourniquet of claim 19, wherein:
said compression strap is formed of Dyneema composite fabric with a length ranging from 32 to 38 inches long and a width ranging between 1¼ to 2 inches.
